(19) United States
(12) Patent Application Publication
(10) Pub. No.: US 2013/0217870 A1
(43) Pub. Date: Aug. 22, 2013

(54) NUCLEIC ACIDS AND METHODS FOR DETECTING TURFGRASS PATHOGENIC FUNGI

(71) Applicants: Nicola Di Maiuta, Zuchwil (CH); Patrick Schwarzentruber, Habsburg (CH)

(72) Inventors: Nicola Di Maiuta, Zuchwil (CH); Patrick Schwarzentruber, Habsburg (CH)

(21) Appl. No.: 13/721,581

(22) Filed: Dec. 20, 2012

Related U.S. Application Data
(62) Division of application No. 12/737,006, filed on Feb. 10, 2011.

(51) Int. Cl. C12Q 1/68

(52) U.S. Cl. CPC C12Q 1/6895
USPC 536/24.33

(57) ABSTRACT

The present invention relates to the use of at least one nucleic acid comprising or consisting of:

(i)
(SEQ ID NO: 1)
CATCGATGAAGAACGCWGCRAAHTGCGATAMGTARTGYGAATTGCAGR
ATTCAGTGARTCATCGAAWYTTTGAACGCAYMTTGCRC,

wherein:
R represents A or G
Y represents C or T
M represents A or C
W represents A or T
H represents A or C or T
(ii) a portion of SEQ ID NO: 1, provided said nucleic acid binds under stringent conditions to a nucleic acid comprising or consisting of the complementary sequence of SEQ ID NO: 1, or
(iii) complementary sequences of (i) and (ii);for the detection of nucleic acids from one or more fungi in a sample.

### CROSS-REFERENCE TO RELATED APPLICATIONS

The present application is a divisional of U.S. application Ser. No. 12/737,006, filed Dec. 1, 2010, which is a U.S. National Phase of PCT Application No. PCT/EP2009/056189, filed May 20, 2009, which claims the benefit of EP Application No. 08104225.1 and U.S. Provisional Application No. 61/059,862, which are incorporated herein by reference.

### FIELD OF THE INVENTION

The present invention relates to nucleic acids and methods for detecting organic substrate pathogenic fungi.

### BACKGROUND OF THE INVENTION

Disease in lawngrasses or turfgrasses develops from an interaction among a susceptible plant, an environment favorable for disease development, and a pathogenic organism, usually a fungus. Such fungi may also develop on decorative grasses, plants and crops; indeed, they may appear on any suitable organic substrate. Thus, treatment of a diseased substrate, especially turfgrass, usually consists in applying fungicides that will either kill the fungus or keep it from growing.

However, the first step in disease management, and especially turfgrass disease management, should always consist in identifying the causative pathogenic agent. Indeed, it is important to have identified the disease correctly, so that an appropriate fungicide can be selected. Arbitary selection and application of fungicides without knowledge of the disease cause can do as much harm as good. Using the wrong fungicide wastes money and may involve the risk of exacerbating the disease, as well as causing other unwanted side effects.

Classical methods for the identification of the causative pathogenic agent essentially rely on the symptoms which can be observed on the individual plant and on the turf stand, as well as on the fungal structures, such as mycelia or spores, which can be found in the vicinity of the diseased turfgrass.

However, these methods may require a long time to be implemented, since they often involve the isolation and the culture of the fungi in a laboratory. Besides, differentiating closely related fungal species can prove difficult.

Accordingly, molecular biology methods have been developped which circumvent these difficulties. One of the most popular fungal detection methods relies on the PCR amplification of the internal transcribed spacers (1, 2) and the 5.8S rRNA gene (ITS1-5.8S-ITS2) from the fungal rRNA operon (Goodwin et al. (1995) Plant Pathology 44:384-391; Ranjard et al. (2001) Applied and Environmental Microbiology 67:4479-4487). However, a specific primer pair is often necessary for the indentification of a given fungal species, which renders this method cumbersome where the identity of pathogenic fungus is unknown and is sought for. Furthermore, some fungal species can not be differentiated using the primers currently available which target this region. Accordingly, this method is not used in routine for determining the antifungal agent most adapted to treat a given turfgrass disease.

Japanese patent application No. 2008005760 discloses 458 probes for detecting molds that can be found in food. These 458 probes are designed for detecting molds by a hybridization-based method involving the use of a microarray.

It is therefore an object of the invention to provide a method which allows the rapid specific detection of nucleic acids from the fungi most commonly involved in turfgrass diseases.

### SUMMARY OF THE INVENTION

The present invention arises from the identification, by the inventors, of a conserved region within the rRNA operon of the genome of pathogenic fungi generally affecting turfgrasses, but also possibly other organic substrates, which is liable to be used as a target in the frame of nucleic acid amplification-based detection method of most of such pathogenic fungi.

Thus the present invention relates to the use of at least one nucleic acid comprising or consisting of:

(i)
(SEQ ID NO: 1)
CATCGATGAAGAACGCWGCRAAHTGCGATAMGTARTGYGAATTGCAGR
ATTCAGTGARTCATCGAAWYTTTGAACGCAYMTTGCRC,

wherein:

R represents A or G

Y represents C or T

M represents A or C

W represents A or T

H represents A or C or T
(ii) a portion of SEQ ID NO: 1, provided said nucleic acid binds under stringent conditions to a nucleic acid comprising or consisting of the complementary sequence of SEQ ID NO: 1, or
(iii) complementary sequences of (i) and (ii);
   for the detection of nucleic acids from one or more fungi in a sample.

In a preferred embodiment, the present invention more particularly relates to the use of at least one nucleic acid comprising or consisting of:
(i) GTGARTCATCGAAWYTTTGAACGCA (SEQ ID NO: 2), wherein:

R represents A or G

Y represents C or T

W represents A or T
(ii) a portion of SEQ ID NO: 2, provided said nucleic acid binds under stringent conditions to a nucleic acid comprising or consisting of the complementary sequence of SEQ ID NO: 1, or
(iii) complementary sequences of (i) and (ii);
   for the detection of nucleic acids from one or more fungi in a sample.

The present invention also relates to a method for detecting nucleic acids from one or more fungi in a sample, wherein at least one nucleic acid comprising or consisting of:

(i)
(SEQ ID NO: 1)
CATCGATGAAGAACGCWGCRAAHTGCGATAMGTARTGYGAATTGCAGR
ATTCAGTGARTCATCGAAWYTTTGAACGCAYMTTGCRC,

wherein:

R represents A or G

Y represents C or T

M represents A or C

W represents A or T

H represents A or C or T
(ii) a portion of SEQ ID NO: 1, provided said nucleic acid binds under stringent conditions to a nucleic acid comprising or consisting of the complementary sequence of SEQ ID NO: 1, or
(iii) complementary sequences of (i) and (ii);
   is contacted with the sample.

In a preferred embodiment, the invention more particularly relates to a method for detecting nucleic acids from one or more fungi in a sample, wherein at least one nucleic acid comprising or consisting of:
(i) GTGARTCATCGAAWYTTTGAACGCA (SEQ ID NO: 2), wherein:

R represents A or G

Y represents C or T

W represents A or T
(ii) a portion of SEQ ID NO: 2, provided said nucleic acid binds under stringent conditions to a nucleic acid comprising or consisting of the complementary sequence of SEQ ID NO: 1, or
(iii) complementary sequences of (i) and (ii);
   is contacted with the sample.

The present invention also relates to a method for treating a diseased organic substrate, which comprises the steps of:
a) detecting the absence or the presence of nucleic acids from at least one pathogenic fungus in a sample of the substrate, with at least one nucleic acid according to the invention as defined above;
b) if nucleic acids from one or more pathogenic fungi have been detected in step a), selecting one or more antifungal agents which target the one or more pathogenic fungi from which nucleic acids have been detected;
c) applying the selected one or more antifungal agents of step b) to the diseased substrate.

The present invention also relates to a kit for the detection of fungi, comprising each one of the nucleic acids represented by:

(i)
(SEQ ID NO: 1)
CATCGATGAAGAACGCWGCRAAHTGCGATAMGTARTGYGAATTGCAGR
ATTCAGTGARTCATCGAAWYTTTGAACGCAYMTTGCRC,

wherein:

R represents A or G

Y represents C or T

M represents A or C

W represents A or T

H represents A or C or T
(ii) a portion of SEQ ID NO: 1, provided said nucleic acid binds under stringent conditions to a nucleic acid comprising or consisting of the complementary sequence of SEQ ID NO: 1, or
(iii) complementary sequences of (i) and (ii).

In a preferred embodiment, the invention more particularly relates to a kit for the detection of fungi, comprising each one of the nucleic acids represented by:
(i) GTGARTCATCGAAWYTTTGAACGCA (SEQ ID NO: 2), wherein:

R represents A or G

Y represents C or T

W represents A or T
(ii) a portion of SEQ ID NO: 2, provided said nucleic acid binds under stringent conditions to a nucleic acid comprising or consisting of the complementary sequence of SEQ ID NO: 1, or
(iii) complementary sequences of (i) and (ii).

The present invention also relates to a nucleic acid comprising or consisting of:

(i)
(SEQ ID NO: 1)
CATCGATGAAGAACGCWGCRAAHTGCGATAMGTARTGYGAATTGCAGR
ATTCAGTGARTCATCGAAWYTTTGAACGCAYMTTGCRC,

wherein:

R represents A or G

Y represents C or T

M represents A or C

W represents A or T

H represents A or C or T
(ii) a portion of SEQ ID NO: 1, provided the said nucleic acid binds under stringent conditions to a nucleic acid comprising or consisting of the complementary sequence of SEQ ID NO: 1, or
(iii) complementary sequences of (i) and (ii).

In a preferred embodiment, the invention more particularly relates to a nucleic acid comprising or consisting of:
(i) GTGARTCATCGAAWYTTTGAACGCA (SEQ ID NO: 2), wherein:

R represents A or G

Y represents C or T

W represents A or T
(ii) a portion of SEQ ID NO: 2, provided said nucleic acid binds under stringent conditions to a nucleic acid comprising or consisting of the complementary sequence of SEQ ID NO: 1, or
   complementary sequences of (i) and (ii).

In another preferred embodiment, the invention more particularly relates to a nucleic acid as defined above, comprising or consisting of a sequence selected from the group consisting of SEQ ID NO: 1 to 38, or complementary sequences thereof.

### DETAILED DESCRIPTION OF THE INVENTION

Nucleic acids as intended herein can be of any type, however it is preferred that they be DNA.

“Stringent conditions” can be easily be defined by the man skilled in the art using common knowledge. If necessary, guidance for defining such conditions can be found in numerous textbooks, such as Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York. Preferably, stringent conditions according to the invention are constituted of an annealing temperature of 60° C. carried out in a PCR reaction medium comprising, e.g. 50 mM KCI, 1.5 mM MgCl₂ and 10 mM Tris pH 8.3.

As intended herein a “portion” of nucleic acid preferably comprises a number of nucleotides sufficient to provide for a specific hybridisation to the nucleic acid comprising or consisting of the complementary sequence of SEQ ID NO: 1. Thus, the portion of nucleic acid preferably comprises at least 9 nucleotides, more preferably at least 15 nucleotides, even more preferably at least 20 nucleotides, and most preferably at least 25 nucleotides.

As intended herein, it is preferred that the maximum length of the nucleic acids according to the invention is less than 500 nucleotides.

Preferably, the at least one nucleic acid as defined above is used as a primer in a nucleic acid amplification-based detection method, such as Amplification Fragment Length Polymorphism (AFLP) or Terminal Restriction Fragment Length Polymorphism (TRFLP or T-RFLP).

Nucleic acid amplification-based detection methods are particularly well known to one of skill in the art. Terminal Restriction Fragment Length Polymorphism (TRFLP or T-RFLP), for instance, is notably described by Formey et al. (1997) Applied and Environmental Microbiology 63:4516-4522.

Where the at least one nucleic acid as defined above is used as a primer, it is preferably used in association with at least one other primer. The other primer can be any primer targeting a sequence comprised within the genome of the one or more fungi. However, it is preferred that the other primer targets the 18S rDNA/ITS1 region or the ITS2/28S rDNA region of the one or more fungi.

The fungal rRNA operon comprises the 18S rRNA gene, the Internal Transcribed Spacer 1 (ITS1), the 5.8S rRNA gene, the Internal Transcribed Spacer 2 (ITS2), and the 28S rRNA gene. The 18S rDNA/ITS1 and ITS2/28S rDNA regions thus relate to the regions of the genome of the one or more fungi in the vicinity respectively of the junction of the 18S rRNA gene and ITS1, and of the junction of TIS2 and the 28S rRNA gene. As will be clear to anyone of skill in the art, where the at least one nucleic acid as defined above is used as a forward primer, the primer which targets the 18S rDNA/ITS1 region or the ITS2/28S rDNA region will be a reverse primer, and vice versa. The sequences of the 18S rDNA/ITS1 and ITS2/28S rDNA regions are well known to one of skill in the art and can usually be accessed from public sequence databases. Where sequences of the 18S rDNA/ITS1 and ITS2/28S rDNA regions would not be publicly available for a particular fungus species, they can be routinely sequenced. Besides, it is well within the common knowledge of anyone of skill in the art to select primers within the known sequences.

By way of example of a primer targeting the 18S rDNA/ITS1 region, one can cite the so-called “ITS1-F primer” of sequence TCCGTAGGTGAACCTGCGG (SEQ ID NO: 39). Conversely, by way of example of a primer targeting the ITS2/28S rDNA region one can cite the so-called “ITS4 primer” of sequence TCCTCCGCTTATTGATATGC (SEQ ID NO: 40). Other examples of primers liable to be used with the primers according to the invention include the “ITS5 primer” of sequence GGAAGTAAAAGTCGTAACAAGG (SEQ ID NO: 41) and the “SR6R primer” of sequence AAGWAAAAGTCGTAACAAGG (SEQ ID NO: 42). Still other Examples are available from http://www.biology.duke.edu/fungi/mycolab/primers.htm.

As intended herein the primers to be used may be unmodified or modified nucleic acids, in particular DNA. Where the primers are modified nucleic acids they can notably be labelled nucleic acids, in particular fluorescently labelled nucleic acids.

Where TRFLP is used, the one of skill in the art knows how to design primers and to select restriction enzymes so that the nucleic acid generated and fragmented by the enzyme presents a fragment length that can be detected. In this regard, it should be noted that detection of a fragment is dependant on the instrumentation and detection techniques used, but in general detection is possible for sequences featuring at least 15 nucleotides. Besides, the length of the fragment should preferably be such that it is distinguishable from fragment lengths generated by the same primer pair/enzyme in other fungi (in general, fragment lengths differing by more than 1, and preferably more than 2, nucleotides are distinguished by current techniques). Numerous databases and tools are available to one of skill in the art for selecting primers and restriction enzymes, such as the REBASE database.

In the case where nucleic acids according to the invention include nucleotides featuring a “wobble” position, that is to say they include nucleotides that may be selected from among two or more possible nucleotides, one of skill in the art knows that it is usually advantageous to use a mixture of primers such that all of the nucleotide possibilities are represented.

Thus, by way of example, each one of the nucleic acids represented by GTGARTCATCGAAWYTTTGAACGCA (SEQ ID NO: 2), are as follows:

(SEQ ID NO: 3)
GTGAATCATCGAAACTTTGAACGCA;
(SEQ ID NO: 4)
GTGAGTCATCGAAACTTTGAACGCA;
(SEQ ID NO: 5)
GTGAATCATCGAATCTTTGAACGCA;
(SEQ ID NO: 6)
GTGAGTCATCGAATCTTTGAACGCA;
(SEQ ID NO: 7)
GTGAATCATCGAAATTTTGAACGCA;
(SEQ ID NO: 8)
GTGAGTCATCGAAATTTTGAACGCA;
(SEQ ID NO: 9)
GTGAATCATCGAATTTTTGAACGCA;
(SEQ ID NO: 10)
GTGAGTCATCGAATTTTTGAACGCA;
(SEQ ID NO: 11)
GTGAATCATCGAAACTTTGAACGCA;
(SEQ ID NO: 12)
GTGAGTCATCGAAACTTTGAACGCA;
(SEQ ID NO: 13)
GTGAATCATCGAATCTTTGAACGCA;
(SEQ ID NO: 14)
GTGAGTCATCGAATCTTTGAACGCA;
(SEQ ID NO: 15)
GTGAATCATCGAAATTTTGAACGCA;
(SEQ ID NO: 16)
GTGAGTCATCGAAATTTTGAACGCA;
(SEQ ID NO: 17)
GTGAATCATCGAATTTTTGAACGCA;
(SEQ ID NO: 18)
GTGAGTCATCGAATTTTTGAACGCA.

As intended herein, it is to be understood that the invention aims at the detection of nucleic acids of fungi of any type, however it is preferred that the fungus is a plant pathogenic fungus, in particular a turfgrass pathogenic fungus, such as a fungus selected from the group constituted of Ascochyta phleina, Curvularia affinis, Glomerella graminicola, Thanatephorus cucumeris, Pythium ultimum, Gaeumannomyces graminis, Marasmius oreades, Corticium fuciforme, Phytophthora nicotianae. Fusarium culmorum, Bipolaris sorokiniana, Microdochium nivale, Rhizoctonia cerealis, Pythium graminicola, Rhynchosporium secalis, Sclerotinia homoeocarpa, Typhula incarnate, Ustilago striiformis, Septoria macropoda.

As intended herein the sample in which nucleic acids are to be detected can be of any type of organic substrate liable to contain nucleic acids from fungi. However, it is preferred that the sample be a turfgrass sample or a soil sample.

Where the sample is a turfgrass sample, it can be a sample obtained from the turfgrass as a whole or from or a sample of a part of the turfgrass, such as the root.

Where the sample is a soil sample, it is preferably taken directly under the diseased turfgrass or in the vicinity of the diseased turgrass.

The sample can be obtained directly from turfgrass or soil, or be obtained after treatment steps, such as grinding or extraction, in particular nucleic acid extraction, steps.

As intended herein, any diseased turfgrass can be subjected the use or methods as defined above. Preferred turfgrasses to be considered within the frame of the present invention are notably described in http://www.ars-grin.gov/cgi-bin/npgs/html/index.pl, from the Germplasm Resources Information Network, National Germplasm Resources Laboratory, Beltsville, Md., or in the Compendium of Turgrass Diseases, Third Edition (2005) by the American Phytopathological Society. Most preferably, the diseased turfgrass according to the invention is selected from the group consisting of the Festaceae, Aveneae, Triticeae, Chlorideae, Zoysieae, Paniceae and Andropogoneae Tribe.

In a specific embodiment, nucleic acids comprising or consisting of the sequences of SEQ ID Nos. 159, 188, 198, 221, 263 and/or 416 of Japanese patent application No. 2008005760 are excluded from the scope of the present invention.

### EXAMPLE

The following turfgrass pathogenic fungus species were sequenced by the inventors: Ascochyta phleina, Curvularia affinis, Glomerella graminicola, Thanatephorus cucumeris, Pythium ultimum, Gaeumannomyces graminis, Marasmius oreades, Corticium fuciforme, Phytophthora nicotianae. Fusarium culmorum, Bipolaris sorokiniana, Microdochium nivale, Rhizoctonia cerealis, Pythium graminicola, Rhynchosporium secalis, Sclerotinia homoeocarpa, Typhula incarnate, Ustilago striiformis, Septoria macropoda.

Briefly, the well-known sequencing method developed by Fred Sanger—chain termination method—was used using Applied Biosystem BigDye® Terminator Cycle Sequencing v1 or v3.1 chemistry on an Applied Biosystems Genetic Analyzer 3130. The sequences were gathered by using the ITS1 forward primer (ITS1-F, TCCGTAGGTGAACCTGCGG, SEQ ID NO: 39).

The obtained sequences are represented by SEQ ID NO: 19 to 37.

From these sequences a particular consensus sequence could be determined by the inventors:

(SEQ ID NO: 1)
CATCGATGAAGAACGCWGCRAAHTGCGATAMGTARTGYGAATTGCAGRAT
TCAGTGARTCATCGAAWYTTTGAACGCAYMTTGCRC

The discriminative potential of this region was then evidenced by in silico Terminal Restriction Fragment Length Polymorphism (TRFLP or T-RFLP) by using the primer ITSOMYAr (TGCGTTCAAARWTTCGATGAYTCAC, SEQ ID NO: 38, the complementary of SEQ ID NO: 2), which hybridizes to the above consensus sequence, in association with the above ITS1-F primer.

The applied enzymes for the T-RFLP analysis were Taql (T^{▾}CG_{▴}A) and Tsp509I (^{▾}AATT_{▴}).

The following tables show the PCR fragment length of both primer combination ITS1-F/ITS4 (full length ITS) (ITS4, TCCTCCGCTTATTGATATGC, SEQ ID NO: 40) and ITS1/ITSOMYAr (partial ITS length) (Table 1), as well as the corresponding in silico and in vitro digested partial ITS (ITS1/ITSOMYAr) fragments by Taql (Table 2) and Tsp509I (Table 3) (mean and standard deviation (StDev) were calculated from triplicate measurements of two independent PCR assays).

TABLE 1

Fragments [bps]

| | Full ITS length | Partial ITS length |
|---|---|---|
| Fungal name | ITS1/ITS4 | ITS1/ITSOMYAr |
| Ascochyta phleina | 588 | 326 |
| Curvularia affinis | 584 | 316 |
| Glomerella graminicola | 587 | 320 |
| Thanatephorus cucumeris | 710 | 360 |
| Pythium ultimum | 914 | 360 |
| Gaeumannomyces graminis | 558 | 280 |
| Marasmius oreades | 672 | 355 |
| Corticium fuciforme | 716 | 353 |
| Phytophthora nicotianae | 892 | 367 |
| Fusarium culmorum | 545 | 286 |
| Bipolaris sorokiniana | 586 | 309 |
| Microdochium nivale | 556 | 283 |
| Rhizoctonia cerealis | 684 | 348 |
| Pythium graminicola | 872 | 319 |
| Rhynchosporium secalis | 627 | 371 |
| Sclerotinia homoeocarpa | 586 | 320 |
| Typhula incarnata | 825 | 410 |
| Ustilago striiformis | 780 | 380 |
| Septoria macropoda | 540 | 277 |

TABLE 2

| | TaqI fragments [bps] | | |
|---|---|---|---|
| | | measured | |
| Fungal name | in-silico | mean | StDev |
| Ascochyta phleina | 252 | 247.0 | 0.022 |
| Curvularia affinis | 242 | 236.4 | 0.048 |
| Glomerella graminicola | 246 | 239.3 | 0.113 |
| Thanatephorus cucumeris | 286 | 284.4 | 0.123 |
| Pythium ultimum | 286 | 284.0 | 0.043 |
| Gaeumannomyces graminis | 206 | 198.9 | 0.030 |
| Marasmius oreades | 281 | 279.6 | 0.057 |
| Corticium fuciforme | 279 | 275.7 | 0.054 |
| Phytophthora nicotianae | 293 | 290.9 | 0.056 |
| Fusarium culmorum | 212 | 207.9 | 0.033 |
| Bipolaris sorokiniana | 235 | 230.2 | 0.054 |
| Microdochium nivale | 209 | 206.5 | 0.247 |
| Rhizoctonia cerealis | 71 | 67.7 | 0.049 |
| Pythium graminicola | 245 | 242.8 | 0.033 |
| Rhynchosporium secalis | 113 | 108.5 | 0.043 |
| Sclerotinia homoeocarpa | 102 | 98.1 | 0.043 |
| Typhula incarnata | 32 | 29.9 | 0.182 |
| Ustilago striiformis | 31 | 29.6 | 0.130 |
| Septoria macropoda | 162 | 147.2 | 0.161 |

TABLE 3

| | Tsp509I fragments [bps] | | |
|---|---|---|---|
| | | measured | |
| Fungal name | in-silico | mean | StDev |
| Ascochyta phleina | 167 | 163.5 | 0.041 |
| Curvularia affinis | 278 | 272.1 | 0.044 |
| Glomerella graminicola | 207 | 201.2 | 0.034 |
| Thanatephorus cucumeris | 34 | 32.3 | 0.091 |
| Pythium ultimum | 322 | 321.4 | 0.040 |
| Gaeumannomyces graminis | 134 | 125.7 | 0.096 |
| Marasmius oreades | 225 | 223.9 | 0.056 |
| Corticium fuciforme | 315 | 312.8 | 0.042 |
| Phytophthora nicotianae | 108 | 104.1 | 0.026 |
| Fusarium culmorum | 248 | 243.3 | 0.029 |
| Bipolaris sorokiniana | 193 | 189.0 | 0.032 |
| Microdochium nivale | 137 | 133.5 | 0.015 |
| Rhizoctonia cerealis | 310 | 307.6 | 0.032 |
| Pythium graminicola | 64 | 58.6 | 0.048 |
| Rhynchosporium secalis | 102 | 329.7 | 0.030 |
| Sclerotinia homoeocarpa | 175 | 172.0 | 0.066 |
| Typhula incarnata | 34 | 31.9 | 0.112 |
| Ustilago striiformis | 143 | 140.7 | 0.013 |
| Septoria macropoda | 239 | 222.4 | 0.169 |

In general, since the length of the fragments yielded by the ITS1/ITSOMYAr combination, either in AFLP or in T-RFLP, is usually smaller than the corresponding fragment obtained using the ITS1/ITS4 combination, the fragments can be separated with a higher resolution.

Furthermore, it can be seen that the ITS1/ITSOMYAr combination provides, either in AFLP or in T-RFLP, a good alternative to the ITS1/ITS4 combination in AFLP since it notably enables to discriminate between species which discrimination was either impossible or very difficult to carry out with the ITS1/ITS4 combination (e.g. Ascochyta phleina, Curvularia affinis, Glomerella graminicola, Bipolaris sorokiniana, Sclerotinia homoeocarpa).

1. A kit for the detection of fungi, comprising at least one nucleic acid primer comprising or consisting of the sequence:
(i) CATCGATGAAGAACGCWGCRAAHTGCGATAMGTARTGYGAATTGCAG RATTCAGTGARTCATCGAAWYTTTGAACGCAYMTTGCRC (SEQ ID NO: 1), wherein:
R represents A or G
Y represents C or T
M represents A or C
W represents A or T
H represents A or C or T
(ii) a portion of SEQ ID NO: 1, provided said nucleic acid binds under stringent conditions to a nucleic acid comprising or consisting of the complementary sequence of SEQ ID NO: 1, or
(iii) a complementary sequence of (i) or (ii).

2. The kit according to claim 1, wherein the primer comprises or consists of:
(i) GTGARTCATCGAAWYTTTGAACGCA (SEQ ID NO: 2), wherein:
R represents A or G,
Y represents C or T,
W represents A or T,
(ii) a portion of SEQ ID NO: 2, provided said nucleic acid binds under stringent conditions to a nucleic acid comprising or consisting of the complementary sequence of SEQ ID NO: 1, or
(iii) a complementary sequence of (i) or (ii).

3. The kit according to claim 1, comprising one or more of the following nucleic acid sequences as the primer(s):
(SEQ ID NO: 3)
GTGAATCATCGAAACTTTGAACGCA;
(SEQ ID NO: 4)
GTGAGTCATCGAAACTTTGAACGCA;
(SEQ ID NO: 5)
GTGAATCATCGAATCTTTGAACGCA;
(SEQ ID NO: 6)
GTGAGTCATCGAATCTTTGAACGCA;
(SEQ ID NO: 7)
GTGAATCATCGAAATTTTGAACGCA;
(SEQ ID NO: 8)
GTGAGTCATCGAAATTTTGAACGCA;
(SEQ ID NO: 9)
GTGAATCATCGAATTTTTGAACGCA;
(SEQ ID NO: 10)
GTGAGTCATCGAATTTTTGAACGCA;
(SEQ ID NO: 11)
GTGAATCATCGAAACTTTGAACGCA;
(SEQ ID NO: 12)
GTGAGTCATCGAAACTTTGAACGCA;
(SEQ ID NO: 13)
GTGAATCATCGAATCTTTGAACGCA;
(SEQ ID NO: 14)
GTGAGTCATCGAATCTTTGAACGCA;
(SEQ ID NO: 15)
GTGAATCATCGAAATTTTGAACGCA;
(SEQ ID NO: 16)
GTGAGTCATCGAAATTTTGAACGCA;
(SEQ ID NO: 17)
GTGAATCATCGAATTTTTGAACGCA;
(SEQ ID NO: 18)
GTGAGTCATCGAATTTTTGAACGCA;
or their complementary sequences.

4. The kit according to claim 1, comprising or consisting of a sequence selected from the group consisting of SEQ ID NO: 1 to 38, or complementary sequences thereof.

5. A nucleic acid comprising or consisting of:
(i) CATCGATGAAGAACGCWGCRAAHTGCGATAMGTARTGYGAATTGCAG RATTCAGTGARTCATCGAAWYTTTGAACGCAYMTTGCRC (SEQ ID NO: 1), wherein:
R represents A or G
Y represents C or T
M represents A or C
W represents A or T
H represents A or C or T
(ii) a portion of SEQ ID NO: 1, provided the said nucleic acid binds under stringent conditions to a nucleic acid comprising or consisting of the complementary sequence of SEQ ID NO: 1, or
(iii) a complementary sequence of (i) or (ii).

6. A nucleic acid according to claim 5, comprising or consisting of a sequence selected from the group consisting of SEQ ID NO: 1 to 38, or complementary sequences thereof.


SEQUENCE LISTING (42 sequences)

SEQ ID NO: 1 - DNA, 86 bp, Artificial Sequence
  Fungal consensus sequence
catcgatgaagaacgcwgcraahtgcgatamgtartgygaattgcagrattcagtgartcatcgaawytttgaacgcaymttgcrc

SEQ ID NO: 2 - DNA, 25 bp, Artificial Sequence
  Degenerated PCR primer for fungal detection
gtgartcatcgaawytttgaacgca

SEQ ID NO: 3 - DNA, 25 bp, Artificial Sequence
  PCR primer for fungal detection
gtgaatcatcgaaactttgaacgca

SEQ ID NO: 4 - DNA, 25 bp, Artificial Sequence
  PCR primer for fungal detection
gtgagtcatcgaaactttgaacgca

SEQ ID NO: 5 - DNA, 25 bp, Artificial Sequence
  PCR primer for fungal detection
gtgaatcatcgaatctttgaacgca

SEQ ID NO: 6 - DNA, 25 bp, Artificial Sequence
  PCR primer for fungal detection
gtgagtcatcgaatctttgaacgca

SEQ ID NO: 7 - DNA, 25 bp, Artificial Sequence
  PCR primer for fungal detection
gtgaatcatcgaaattttgaacgca

SEQ ID NO: 8 - DNA, 25 bp, Artificial Sequence
  PCR primer for fungal detection
gtgagtcatcgaaattttgaacgca

SEQ ID NO: 9 - DNA, 25 bp, Artificial Sequence
  PCR primer for fungal detection
gtgaatcatcgaatttttgaacgca

SEQ ID NO: 10 - DNA, 25 bp, Artificial Sequence
  PCR primer for fungal detection
gtgagtcatcgaatttttgaacgca

SEQ ID NO: 11 - DNA, 25 bp, Artificial Sequence
  PCR primer for fungal detection
gtgaatcatcgaaactttgaacgca

SEQ ID NO: 12 - DNA, 25 bp, Artificial Sequence
  PCR primer for fungal detection
gtgagtcatcgaaactttgaacgca

SEQ ID NO: 13 - DNA, 25 bp, Artificial Sequence
  PCR primer for fungal detection
gtgaatcatcgaatctttgaacgca

SEQ ID NO: 14 - DNA, 25 bp, Artificial Sequence
  PCR primer for fungal detection
gtgagtcatcgaatctttgaacgca

SEQ ID NO: 15 - DNA, 25 bp, Artificial Sequence
  PCR primer for fungal detection
gtgaatcatcgaaattttgaacgca

SEQ ID NO: 16 - DNA, 25 bp, Artificial Sequence
  PCR primer for fungal detection
gtgagtcatcgaaattttgaacgca

SEQ ID NO: 17 - DNA, 25 bp, Artificial Sequence
  PCR primer for fungal detection
gtgaatcatcgaatttttgaacgca

SEQ ID NO: 18 - DNA, 25 bp, Artificial Sequence
  PCR primer for fungal detection
gtgagtcatcgaatttttgaacgca

SEQ ID NO: 19 - DNA, 589 bp, Ascochyta phleina
tccgtaggtgaacctgcggaaggatcattacactcagtagtttactactgtaaaggaggccgtcagtctgtatagcttcatcgctgacgagcagctggtctcttttatccacccttgtcttttgcgtacccatgtttcctcggcaggcttgcctgccgactggacaaatttataacctttttaattttcaatcagcgtctgaaaaacttaataattacaactttcaacaacggatctcttggttctggcatcgatgaagaacgcagcgaaatgcgataagtagtgtgaattgcagaattcagtgaatcatcgaatctttgaacgcacattgcgccccttggtattccatggggcatgcctgttcgagcgtcatttgtaccctcaagctttgcttggtgttgggtgtttgtcctctccctggtgtttggactcgccttaaaacaattggcagccagtgttttggtattgaagcgcagcacaagtcgcgattctaacctagtaacacttgcgtccacaagccttttttcacttttgacctcggatcaggtagggatacccgctgaacttaagcatatcaataagcggagga

SEQ ID NO: 20 - DNA, 584 bp, Curvularia affinis
tccgtaggtgaacctgcggagggatcattacacaataaacatatgaaggctgcaccgccagttggcggcaaggctggagtattttattacccttgtcttttgcgcacttgttgtttcctgggcgggttcgcccgccaccaggaccacatgataaaccttttttatgcagttgcaatcagcgtcagtacaacaaatgtaaatcatttacaactttcaacaacggatctcttggttctggcatcgatgaagaacgcagcgaaatgcgatacgtagtgtgaattgcagaattcagtgaatcatcgaatctttgaacgcacattgcgccctttggtattccaaagggcatgcctgttcgagcgtcatttgtaccctcaagctttgcttggtgttgggcgttttgtctttggttgccaaagactcgccttaaaacgattggcagccggcctactggtttcgcagcgcagcacatttttgcgcttgcaatcagcaaaagaggacggcactccatcaagactctttatcacttttgacctcggatcaggtagggatacccgctgaacttaagcatatcaataagcggagga

SEQ ID NO: 21 - DNA, 587 bp, Glomerella graminicola
tccgtaggtgaacctgcggagggatcattactgagttaccgctctataaccctttgtgaacatacctaaccgttgcttcggcgggttagggggtcccctctccgggggacgccctcccggccgggccccactgcggggctcggcgcccgccggaggataaccaaactctgatttaacgacgtctcttctgagtggcacaagcaaataattaaaacttttaacaacggatctcttggttctggcatcgatgaagaacgcagcgaaatgcgataagtaatgtgaattgcagaattcagtgaatcatcgaatctttgaacgcacattgcgcccgccagcattctggcgggcatgcctgttcgagcgtcatttcaaccctcaagctccgcttggtgttggggccctacggcgtacgtcgtaggcccttaaaggtagtggcggaccctcccggagcctcctttgcgtagtaactaacgtctcgcatcgggatccggagggactcttgccgtaaaacccccaactttttaactggttgacctcggatcaggtaggaatacccgctgaacttaagcatatcaataagcggagga

SEQ ID NO: 22 - DNA, 710 bp, Thanatephorus cucumeris
tccgtaggtgaacctgcggaaggatcattattgaattttattaatgaggagttgagttgttgctggccttttctaccttaatttggcaggagggggcatgtgcacaccttctcttttatccatcacaccccctgtgcacttgtgagacagcaatagttggtggatttaattccatccatttgctgtctacttaatttacacacactctacttaatttaaactgaatgtaattgatgtaacgcatctaatactaagtttcaacaacggatctcttggctctcgcatcgatgaagaacgcagcgaaatgcgataagtaatgtgaattgcagaattcagtgaatcatcgaatctttgaacgcaccttgcgctccttggtattccttggagcatgcctgtttgagtatcatgaaatcttcaaagtaaaccttttgttaattcaattggtcttttttactttggttttggaggatcttattgcagcttcacacctgctcctctttgtgcattagctggatctcagtgttatgcttggttccactcggcgtgataagttatctatcgctgaggacacccgtaaaaaaggtggccaaggtaaatgcagatgaaccgcttctaatagtccattgacttggacaatattctattttatgatctgatctcaaatcaggtaggactacccgctgaacttaagcatatcaataagcggagga

SEQ ID NO: 23 - DNA, 914 bp, Pythium ultimum
tccgtaggtgaacctgcggaaggatcattaccacactttaaaaaactgtccacgtgaactgtaagcaagtctagcgctgtgactgagctggtgttttcatttttggacactggaacgggagtcagcaggacgaaggttggtctgttgtaatgcaagttatgatggactagctgatgaacttttgtttttaaacccttacctaaatactgatttatactgtggggacgaaagtccttgcttttactagataacaactttcagcagtggatgtctaggctcgcacatcgatgaagaacgctgcgaactgcgatacgtaatgcgaattgcagaattcagtgagtcatcgaaattttgaacgcatattgcactttcgggttatgcctggaagtatgtctgtatcagtgtccgtaaatcaaacttgcctttctttttccgtgtagtcagggatggaatgtgcagatgtgaagtgtctcgcatggttgcgttcgttttttcgatcgagaatctgtcgagtccttttaaatggacacggtcttttctatggtttctatgaagtgtaatggttggaaggcagtgattttcggattgctggcggcttttggcgacttcggtatgaacgtatggagactagctcaattcgtggtatgttaggcttcggctcgacaatgttgcgtaattgtgtgtggtctttgtttgtgccttgaggtgtactagaggttgtcggtttgaaccgtaagtgattgtttagtagagcattttcacgatgtatggagacgctgcatttagttgcgtagagagattgatttgggaaattttgtatcattgtcaattgcaagattgtgtatggtatctcaattggacctggtatcagacaagactacccgctgaacttaagcatatcaataagcggagga

SEQ ID NO: 24 - DNA, 558 bp, Gaeumannomyces graminis
tccgtaggtgaacctgcggagggatcattacagagttgaaaaactccaacccctgtgaacatacctttactgttgcttcggcggacgatggccccccccgggggccggacgccgccggaggttacaaaccctgaattttagtgtatctctgagtataaaaccaaataattaaaactttcaacaacggatctcttggttctggcatcgatgaagaacgcagcgaaatgcgataagtaatgtgaattgcagaattcagtgaatcatcgaatctttgaacgcacattgcgcccgccggtattccggcgggcatgcctgtccgagcgtcatttcaccactcaagcccagcttggtgttggggcacccggccgcccggcggtcggggcccccaagaacatcggcggtctcgccaggaccctgaacgcagtaactcgcggtaaaacgcgcttcgttcggaggcttcccggcgggctccagccgctaaaccccctaaacttcttaggttgacctcggatcaggtaggaatacccgctgaacttaagcatatcaataagcggagga

SEQ ID NO: 25 - DNA, 672 bp, Marasmius oreades
tccgtaggtgaacctgcggaaggatcattattgaaacattgtaaaggaaggttgagctggctcttcacgggcatgtgctcgcctttctttcaatcttcatccacctgtgcactttttgtagggagctttgagaatgggacctctcgcggggttcctagtattgggctctctatgtcttcacacactcttgaatgtatgtcgttgaatgtcttttacagggacttaattgaccctttaaaaactatacaactttcagcaacggatctcttggctctcgcatcgatgaagaacgcagcgaaatgcgataagtaatgtgaattgcagaattcagtgaatcatcgaatctttgaacgcaccttgcgcctcttggtattccgagaggcatgcctgtttgagtgtcattaaattctcaacttcaaaagcttttgtttttgaagcttggatgtggaggctttgctggctcttctagagtcggctcctctgaaatgcattagtggaaactgtttgcaatccgcattggtgtgataattatctacgcttgtgtgtggctgcagctctttacgagtttagtatccgcttcaaaccgtcctaagttactggacaacttgaaccttttgacctcaaatcaggtaggactacccgctgaacttaagcatatcaataagcggagga

SEQ ID NO: 26 - DNA, 716 bp, Corticium fuciforme
tccgtaggtgaacctgcggaaggatcattaacgagttttaaaagagttgtagctggccctctggggtatgtgcacgctctactcatccacatacacctgtgcacatagatagtcttttttttgagaagggggagaaaaagtagccctttgggtgaaaagtttcccccctcttcagaaagctattctttttatacacatacactttagttaagaatgtatatattgctataaaacgcattaaatataactttcaacaacggatctcttggctctcgcatcgatgaagaacgcagcgaaatgcgataagtaatgtgaattgcagaattcagtgaatcatcgaatctttgaacgcaccttgcgctccttggtattccgaggagcatgcctgtttgagtgtcatgaatatctcaactctcaaagttctgtaatggatcattgagagcttggactttggaggcttgctggtcatcatgtatcagctcctcttaaatgaattagctctggattacgatgtagtttatctacggtgtgatacatgtctacgctattagataactacgaacgttacaattcctccatttctggggtttgcgtgactagtaaatacaaagcttctaattgtctttcgggacaaaacctgatttcattatcttgacatctgacctcaaatcaggtaggactacccgctgaacttaagcatatcaataagcggagga

SEQ ID NO: 27 - DNA, 892 bp, Phytophthora nicotianae
tccgtaggtgaacctgcggaaggatcattaccacacctaaaaaactttccacgtgaaccgtttcaacccaatagttgggggtcttatttggcggcggctgctggcttaattgttggcggctgctgctgagtgagccctatcaaaaaaaaggcgaacgtttgggcttcggcctgatttagtagtctttttttcttttaaacccattccttaatactgaatatactgtggggacgaaagtctctgcttttaactagatagcaactttcagcagtggatgtctaggctcgcacatcgatgaagaacgctgcgaactgcgatacgtaatgcgaattgcaggattcagtgagtcatcgaaattttgaacgcatattgcacttccgggttagtcctggaagtatgcctgtatcagtgtccgtacattaaacttgactttcttccttccgtgtagtcggtggaggagatgtcagatgtgaagtgtcttgcgattggtcttcggaccggctgcgagtccttttaaatgtactaaactgaacttctctttgctcgaaaagtggtggcgttgctggttgtgaaggctgctattgtggcaaattggcgactggtttgtctgctgcggcgttaatgggagagtgttcgattcgtggtatggttggcttcggctgaacaatgcacttattggacgtttttcctgctgtggcgtgatggactggtgaaccatagctcggtggcttggcttttgaattggctttgctgttgcgaagtagggtggcagcttcggttgtcgagggtcgatccatttgggaacttaatgtgtacttcggtatgcatctcaattggacctgatatcaggcaagattacccgctgaacttaagcatatcaataagcggagga

SEQ ID NO: 28 - DNA, 545 bp, Fusarium culmorum
tccgtaggtgaacctgcggagggatcattaccgagtttacaactcccaaacccctgtgaacataccttatgttgcctcggcggatcagcccgcgccccgtaaaaagggacggcccgccgcaggaaccctaaactctgtttttagtggaacttctgagtataaaaaacaaataaatcaaaacttccaacaacggatctcttggttctggcatcgatgaagaacgcagcaaaatgcgataagtaatgtgaattgcagaattcagtgaatcatcgaatctttgaacgcacattgcgcccgccagtattctggcgggcatgcctgttcgagcgtcatttcaaccctcaagcccagcttggtgttgggagctgcagtcctgctgcactccccaaatacattggcggtcacgtcgagcttccatagcgtagtaatttacatatcgttactggtaatcgtcgcggccacgccgttaaaccccaacttctgaatgttgacctcggatcaggtaggaatacccgctgaacttaagcatatcaataagcggagga

SEQ ID NO: 29 - DNA, 586 bp, Bipolaris sorokiniana
tccgtaggtgaacctgcggagggatcattacacaacaaaatatgaaggcctggcttcgcggccggctgaaatatttttttcacccatgtcttttgcgcacttgttgtttcctgggcgggttcgcccgccaccaggaccaaaccataaaccttttttttatgcagttgcaatcagcgtcagtaaaaacaatgtaattattacaactttcaacaacggatctcttggtcctggcatcgatgaagaacgcagcgaaatgcgatacgtagtgtgaattgcagaattcagtgaatcatcgaatctttgaacgcacattgcgccctttggtattccaaagggcatgcctgttcgagcgtcatttgtaccttcaagctttgcttggtgttgggcgttttttgtctccctctttctgggagactcgccttaaaacgattggcagccggcctactggtttcggagcgcagcacatattttgcgctttgtatcaggagaaaaggacggtaatccatcaagactctacatttttaacttttgacctcggatcaggtagggatacccgctgaacttaagcatatcaataagcggagga

SEQ ID NO: 30 - DNA, 556 bp, Microdochium nivale
tccgtaggtgaacctgcggagggatcattactgagtttttaactctccaaaccatgtgaacttaccactgttgcctcggtggatggtgctgtctctcgggacggtgccaccgccggtggactacctaaactctgttaatttttgtcaatctgaatcaaactaagaaataagttaaaactttcaacaacggatctcttggttctggcatcgatgaagaacgcagcgaaatgcgataagtaatgtgaattgcagaattcagtgaatcatcgaatctttgaacgcacattgcgcccattagtattctagtgggcatgcctgttcgagcgtcatttcaacccttaagcctagcttagtgttgggagactgcctaatacgcagctcctcaaaaccagtggcggagtcggttcgtgctctgagcgtagtaattttttatctcgcttctgcaagccggactggcaacagccataaaccgcacccttcgggggcactttttaatggttgacctcggatcaggtaggaatacccgctgaacttaagcatatcaataagcggagga

SEQ ID NO: 31 - DNA, 684 bp, Rhizoctonia cerealis
tccgtaggtgaacctgcggaaggatcattagtgaatgaatgtagagtcggttgtagctgggtcttttaatcgaggccatgtgcacgccttctctttcatccacacacacctgtgcacctgtttagacggtcgaaggaaaaagtctttctcgcgagagagaggctggctccttttccgtccaatacataaaatcttatatatttaatcagaatgtaatcgatgtaaacgcatctataaactaagtttcaacaacggatctcttggctctcgcatcgatgaagaacgcagcgaaatgcgataagtaatgtgaattgcagaattcagtgaatcatcgaatctttgaacgcaccttgcgctccttggtattcctcggagcacgcctgtttgagtatcatgaaattctcaaagcaagtcttttgttaattcaactggcttttgttttggatttggaggttttgcagattcacgtctgctcctcttaaatgcattagctggatctctataaaaccggttccactcggcgtgataagtatcactcgctgaggacactcttgaaaaagggtggccggattcatggatgaaccgcttctaacggtctattagattagacaaacacactttatgatctgatctcaaatcaggtgggactacccgctgaacttaagcatatcaataagcggagga

SEQ ID NO: 32 - DNA, 872 bp, Pythium graminicola
tccgtaggtgaacctgcggaaggatcattaccacaccaaaaaactttccacgtgaaccgttataattatgttctgtgctttccttcgggaaggctgaacgaaggttgatcgtatgtattaatttatgtgtggtcttccgatgtcttttaaacccattacttaatactgatctatactccgagaacgaaagtttttggttttaatccataacaactttcagcagtggatgtctaggctcgcacatcgatgaagaacgctgcgaactgcgatacgtaatgcgaattgcagaattcagtgagtcatcgaaattttgaacgcacattgcactttcgggatattcctggaagtatgcttgtatcagtgtccgtacatcaaacttgcctttctttttttgtgtagtcaaggagagaaatggccgattgtgaggtgtctcgttgactcccttttcggaggagaagacgcgagtccctttaaatgtacgttcgctctttcttgtgtctgaggtgaagtgtgactttcgaacgcattgatctgtttggatcgttttgcgcgagtgggcgacttcggttaggacgttaaaggaagcaaccattattggcggtatgttagacttcggtccgactttgcagctgagagtgtgtagttttctgttctttccttgaggtgtacctgtttgtgtgaggcaatggtctgagcaaatggttattgtgtagtagaattttgctgctcttggacgccctattcgtagggtaaagtagacaacaccattttgggactagtctatgtattaatttatgtgggcgatttttcaatttggacctgatatcaagtaagactacccgctgaacttaagcatatcaataagcggagga

SEQ ID NO: 33 - DNA, 627 bp, Rhynchosporium secalis
tccgtaggtgaacctgcggaaggatcattaatagagcaaagaacagtcagcgccctaggagaaatcccgggggctaccctacttcggtggggtttagagacgtcaggccgctcgaagaagcctggttcagacctccacccttgaataaactacctttgttgctttggcaggccgcccagcgccagcggcttcggctgctgagtgcctgccagaggaccacaactcttgtttttagtgatgtctgagtactatataatagttaaaactttcaacaacggatctcttggttctggcatcgatgaagaacgcagcgaaatgcgataagtaatgtgaattgcagaattcagtgaatcatcgaatctttgaacgcacattgcgccctctggtattccggggggcatgcctgttcgagcgtcattataaccactcaagctctcgcttggtattggagttcgcgtcctcgcggcccctaaaatcagtggcggtgcctgtcggctctacgcgtagtaatactcctcgcgattgagtccggctggtctacttgccaataacccccaaatttttacaggttgacctcggatcaggtagggatacccgctgaacttaagcatatcaataagcggagga

SEQ ID NO: 34 - DNA, 586 bp, Sclerotinia homoeocarpa
tccgtaggtgaacctgcggaaggatcattaccgagttcacgccctcacgggtagacctccaacccttgtgtatctctaccatgttgctttggcaggctgctcgacccttccggggacagcctcagcgccctccggggccggagagtcgcctgccggaggaaaatcacaactctgaattgtcagtgtcgtctgagtgactatctaatagttaaaactttcaacaacggatctcttggttctggcatcgatgaagaacgcagcgaaatgcgataagtaatgtgaattgcagaattcagtgaatcatcgaatctttgaacgcacattgcgccccttggtattccggggggcatgcctgttcgagcgtcatttcaaccctcaagctctctgcttggtattgggcctccgccggtcacacggcgggccttaaagtcagtggcggcgccgctgggtcctgaacgtagtaacacatacctctcgttacagggtccccgcgcgctcccgccgtaaaacccccctcattttctctggttgacctcggatcaggtagggatacccgctgaacttaagcatatcaataagcggagga

SEQ ID NO: 35 - DNA, 825 bp, Typhula incarnate
tccgtaggtgaacctgcggaaggatcattatcgaatttaaggctttggttgagctggcgcttcggtgcatgtgcttgccttgtgctgtccattcttcaacacctgtgcacactttgtagttgactcttttgtttatctgttcatctctctcttgactccggtctctgtgaaggggtgcgtggcttttcgaaagcaaggtcctctatgttattattatacaccctttacaaaaaacaagtccatagaacgtccaatgtaggcgcagcgtaaaacactgtttgctgaaattataaaacttatacaacttttaacaacggatctcttggctctcgcatcgatgaagaacgcagcgaaatgcgataagtaatgtgaattgcagaattcagtgaatcatcgaatctttgaacgcaccttgcgctccctggtattccggggagcatgcctgtttgagtgtcattaaattctcaaccacactatgtttttattaacgtagttcttgtggcttggatcttggagtttgtgccggtaaacctttagttaggttgttggctcctctttaaatgcattagctggaacctctttgtggtgccagactatggtgtgataattatctacgctgtggtttgttgcgctgcgaatttaactatggggttctgcttctaatcgtccctttcaaaggacagtatagagtgtggtggggtgggggttgctttcaaggggttcgcctcttgtgttacaatcttgccctttacctcttaattgacattttgacctcaaatcaggtaggactacccgctgaacttaagcatatcaataagcggagga

SEQ ID NO: 36 - DNA, 780 bp, Ustilago striiformis
tccgtaggtgaacctgcggatggatcatttcgatgaaaacctttttttcttgaggtgtggctcgcacctgtctaactaaacttgagctaccttttttcaacacggttgcatcggttggcctgtcaaacagtgcggcggcgtgaattttcacgtctgctttggctgggcgacggaccgacacttaatcaacacttttgataatctaggatttgaatgataaaagttcatttttacaatgaaatcgactggtaatgcggtcgtctaatttttaaaaacaacttttggcaacggatctcttggttctcccatcgatgaagaacgcagcgaattgcgataagtaatgtgaattgcagaagtgaatcatcgaatctttgaacgcaccttgcgctcccggcagatctaatctggggagcatgcctgtttgagggccgcgaattgtttcgaacgacaactttttctctcttttttttgaagagttggcggatcggtattgagggttttgccattcaccgtggctccctcgaaatgcattagcgcatccatttgataggcaagacggacgaaagctcgattttcgctctctcttccctgccgggtttcgataatatcaggacttcggagaggttgagatgggtaagagctggacgcaacggcttgctgtttggagtgcttctgaaacccgcccatatcgagctttgcctcggaagggactttaataattcatcggcctcagattggtaggactacccgctgaacttaagcatatcaataagcggagga

SEQ ID NO: 37 - DNA, 540 bp, Septoria macropada
tccgtaggtgaacctgcggagggatcattaccgagtgggggcctccgggtccgatctccaaccctttgtgaacacatcccgttgcttcgggggcgcccgggccgggcgcccccggaggaccatccaacactgcatctttgcgtcggagtttacgagtaaatcgaaacaaaactttcaacaacggatctcttggttctggcatcgatgaagaacgcagcgaaatgcgataagtaatgtgaattgcagaattcagtgaatcatcgaatctttgaacgcacattgcgccccctggtattccggggggcatgcccgttcgagcgtcattacaccactccagcctcgctgggtattgggcgtccgcgggggagcaatcccccgcgcgcctcaaagtctccggctgagcggtcccgtctcccagcgttgtggcatcacgtctcgccgcggagtctcgggccctcacggccgttaaatcacacctcaggttgacctcggatcgggtagggatacccgctgaacttaagcatatcaataagcggagga

SEQ ID NO: 38 - DNA, 25 bp, Artificial Sequence
  Degenerated PCR primer for fungal detection
tgcgttcaaarwttcgatgaytcac

SEQ ID NO: 39 - DNA, 19 bp, Artificial Sequence
  ITS1-F primer
tccgtaggtgaacctgcgg

SEQ ID NO: 40 - DNA, 20 bp, Artificial Sequence
  ITS4 primer
tcctccgcttattgatatgc

SEQ ID NO: 41 - DNA, 22 bp, Artificial Sequence
  ITS5 primer
ggaagtaaaagtcgtaacaagg

SEQ ID NO: 42 - DNA, 20 bp, Artificial Sequence
  SR6R primer
aagwaaaagtcgtaacaagg